(19) United States
(12) Patent Application Publication
(10) Pub. No.: US 2019/0068927 A1
(43) Pub. Date: Feb. 28, 2019

(54) OPTICAL FIBER TRANSMISSION LINES FOR ULTRA-SMALL IMAGE SENSORS

(71) Applicant: OmniVision Technologies, Inc., Santa Clara, CA (US)

(72) Inventors: Xiang-Dong Xiong, Milpitas, CA (US); Tiejun Dai, Santa Clara, CA (US); Heng Fan, Palo Alto, CA (US)

(21) Appl. No.: 15/691,678

(22) Filed: Aug. 30, 2017

(51) Int. Cl. H04N 7/22; H04N 5/369; H04N 5/378; H04N 5/376; H04N 7/10; H04N 5/225; G02B 23/24; A61B 1/00; A61B 1/05

(52) U.S. Cl. CPC H04N 7/22; H04N 5/3698; H04N 5/378; H04N 5/3765; H04N 7/10; H04N 5/2251; A61B 1/0684; G02B 23/2469; A61B 1/0017; A61B 1/00013; A61B 1/05; H04N 2005/2255; G02B 23/2484

(57) ABSTRACT

An image sensor system, comprising: an image sensor including an image sensing pixel array; a supporting module; a ground line coupling the image sensor with the supporting module; a power line coupling the image sensor with the supporting module; a data/control line coupling the image sensor with the supporting module; and a clock line coupling the image sensor with the supporting module; wherein the ground line and the power line are based on electrical conduction, and wherein the clock line is based on fiber optics.

### TECHNICAL FIELD

This disclosure relates generally to ultra-small image sensors. In particular, the clock and/or data/control signal transmission lines of an ultra-small image sensor utilize optical fibers to transmit signals digitally. An ultra-small image sensor is a class of image sensors with a product package size of about 1 mm×1 mm, or less. Conventionally, image sensors use conductor-based electrical transmission lines to transmit electrical signals. In the current disclosure, optical fibers are used to replace conventional transmission lines for the clock and/or the data/control signal transmission.

### BACKGROUND INFORMATION

An image sensor uses opto-electronic components, such as photodiodes, to detect incoming light and produce electronic signals in response. A primary component of the image sensor is its sensor pixel array, wherein each pixel includes a photodiode to convert photons to charge carriers, a floating node to temporarily store the charge carries, and a number of transistor gates (transfer gate, source follower, reset transistor, etc.) to convey the charge carriers out of the pixel to be further processed by a peripheral supporting circuitry. An image sensor is often packaged with its supporting module into an image sensor system package, which is then incorporated into a final imaging product such as a mobile phone camera, a consumer electronic camera, a surveillance video camera, an automotive driver assistance system, an industrial imaging borescope, a medical imaging endoscope, etc. The supporting module may provide power and ground connections to the image sensor. Additionally, the supporting module may send control signals, including clock signals, to the image sensor. In return, the image sensor may send image data signals, such as video signals to the supporting module to be further processed into output images. Transmission lines are conventionally used to couple the image sensor with its supporting module.

### BRIEF DESCRIPTION OF THE DRAWINGS

Non-limiting and non-exhaustive examples of the invention are described with reference to the following figures, wherein like reference numerals refer to like parts throughout the various views unless otherwise specified.

FIG. 1 is a schematic drawing of a first embodiment of an ultra-small image sensor system, wherein an image sensor coupled with a supporting module by a number of transmission lines.

FIG. 2 is a drawing that shows a pulse-train clock signal causing noise to an image data signal.

FIG. 3 is a schematic drawing of a second embodiment of an ultra-small image sensor system, wherein an image sensor is coupled with a supporting module by a number of transmission lines, and wherein the clock line is a fiber optic line.

FIG. 4 is a front side view of a third embodiment of an ultra-small image sensor system package.

FIG. 5 is a cross sectional side view of a third embodiment an ultra-small image sensor system package.

Corresponding reference characters indicate corresponding components throughout the several views of the drawings. Skilled artisans will appreciate that elements in the figures are illustrated for simplicity and clarity and have not necessarily been drawn to scale. For example, the dimensions of some of the elements in the figures may be exaggerated relative to other elements to help to improve understanding of various embodiments of the present invention. Also, common but well-understood elements that are useful or necessary in a commercially feasible embodiment are often not depicted in order to facilitate a less obstructed view of these various embodiments of the present invention.

### DETAILED DESCRIPTION

In the following description, numerous specific details are set forth to provide a thorough understanding of the examples. One skilled in the relevant art will recognize; however, that the techniques described herein can be practiced without one or more of the specific details, or with other methods, components, materials, etc. In other instances, well-known structures, materials, or operations are not shown or described in detail to avoid obscuring certain aspects.

Reference throughout this specification to “example” or “embodiment” means that a particular feature, structure, or characteristic described in connection with the example is included in at least one example of the present invention. Thus, the appearances of “example” or “embodiment” in various places throughout this specification are not necessarily all referring to the same example. Furthermore, the particular features, structures, or characteristics may be combined in any suitable manner in one or more examples.

Throughout this specification, several terms of art are used. These terms are to take on their ordinary meaning in the art from which they come, unless specifically defined herein or the context of their use would clearly suggest otherwise.

First Image Sensor System Embodiment

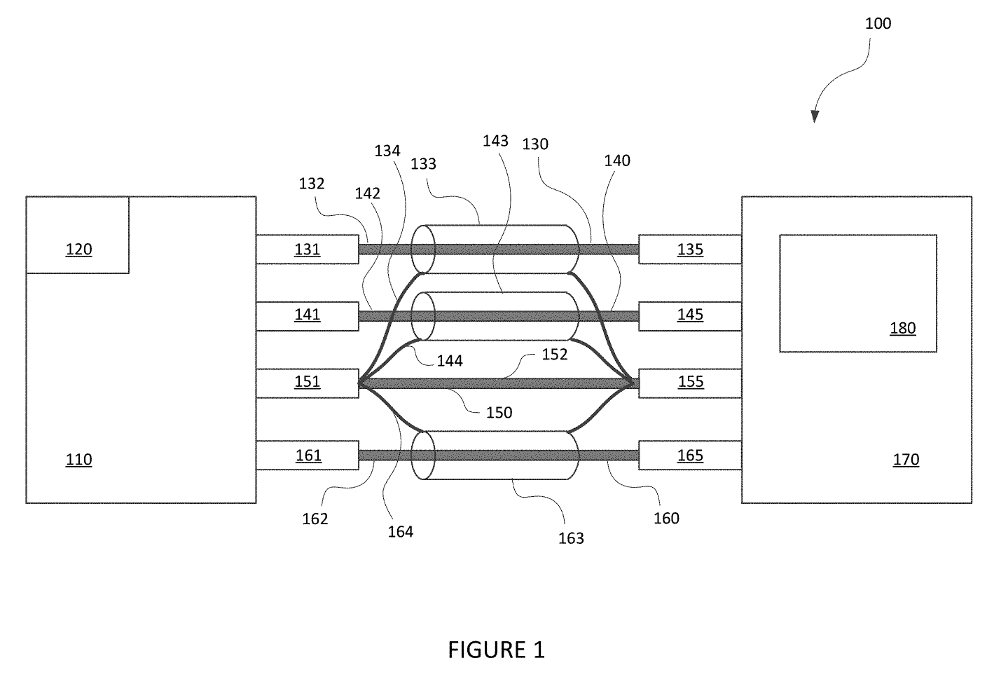

FIG. 1 is a schematic drawing of a first embodiment of an ultra-small image sensor system 100, which includes an ultra-small image sensor 110 that is coupled to a supporting module 170. The ultra-small image sensor 110 has an exemplary package width of about 1 mm×1 mm, e.g., 1.0±0.3 mm in diameter or diagonal dimension. It includes an image sensing member 120 that is, for example, an array of image sensing pixels. Each pixel in this pixel array includes a photodiode to convert photons to charge carriers, a floating node to temporarily store the charge carries, and a number of transistor gates (transfer gate, source follower, reset transistor, etc.) to convey the charge carriers out of the pixel to be further processed by a peripheral supporting circuitry, which is part of the image sensor 110 that surrounds the image sensing pixel array 120.

Due to the ultra-small dimension of the image sensor 110, many of its control and data signal processing function need to be performed elsewhere outside the sensor, for example, by the supporting module 170, which may perform as a controller, a power source, and other supporting functions. More particularly, the supporting module 170 may include a bridge 180, which includes functional blocks such as analog-digital converter (ADC), decoder, image signal processor (ISP), etc. To maintain the ultra-small dimension of the image sensor 110, its internal circuitry may be minimized. For example, only circuits directly relating to image sensing are kept inside the image sensor 110. Circuits performing other functions are placed outside the image sensor 110. For example, they are placed inside the supporting module 170. In addition, the number of transmission lines that couple the image sensor 110 with the supporting module 170 is minimized to be only four lines. Specifically, there is a power line 140, a ground line 150, a data/control signal line 130, and a clock line 160. The ultra-small dimension of the image sensor 110 entails that only four transmission lines may be accommodated by it. There is no room for additional transmission lines going into the image sensor 110.

FIG. 1 shows specifically that, for the ground line 150, the image sensor 110 includes a sensor-side ground line coupler 151, and the supporting module 170 includes a module-side ground line coupler 155. The ground line 150 connects the two couplers 151 and 155, and is essentially a conductor line 152 that may be made of an electrically conductive material such as copper, or other highly conductive metals.

FIG. 1 also shows that, for the data/control signal transmission line 130, the image sensor 110 includes a sensor-side data/signal line coupler 131, and the supporting module 170 includes a module-side data/signal line coupler 135. The data/control signal line 130 connects the two couplers 131 and 135. The data/control signal line 130 is shown to be a co-axial cable that includes a conductive line core 132 and a line shield 133. Transmission line types other than the co-axial cable may also be viable, e.g., a twisted pair line. The shield 133 is connected to the ground line 150 with one or a number of shield grounding lines 134. This grounding is desirable, but it is also optional.

Similarly, for the power line 140 and the clock line 160, the image sensor 110 includes a sensor-side power line coupler 141 and a sensor-side clock line coupler 161, respectively; the supporting module 170 includes a module-side power line coupler 145 and a module-side clock line coupler 165, respectively. The power line 140 connects the two power line couplers 141 and 145; the clock line 160 connects the two clock line couplers 161 and 165. Both the power line 140 and the clock line 160 are shown to be co-axial cables that each includes a conductive line core 142, 164; and a line shield 143, 163. Transmission line types other than the co-axial cable may also be viable, e.g., a twisted pair line. Each shield 143, 163 is connected to the ground line 150 with one or a number of shield grounding lines 144, 164, respectively. This grounding is desirable, but it is also optional.

All the sensor-side couplers 131, 141, 151, and 161 are connected to the internal circuit that is inside the image sensor 110. All the module-side couplers 135, 145, 155, and 165 are connected to the circuit that is inside the supporting module 170.

The ground line 150 provides an electrical grounding function. The data/control signal line 130 conveys image data signal from the image sensor 110 to the supporting module 170. It may also convey sensor control signals from the supporting module 170 to the image sensor 110. The power line 140 supplies electrical power from the supporting module 170 to the image sensor 110. The clock line 160 supplies clock signal from the supporting module 170 to the image sensor 110, as a necessary part for the timing operation of the sequential logic circuits within the image sensor system 100.

Noise Caused by a Pulse-Train Clock Signal

A major problem of the ultra-small image sensor system 100 is electronic noise caused by the clock transmission line 160. Every image sensor system requires a timing operation, so a clock line is a routine component of such an image sensor system. Clock line related noise is generally not an issue for regular size image sensors, whose width is about 2 mm or more. However, in an ultra-small image sensor, the circuit components are positioned very close to each other, so some circuit components are now more susceptible to noise generated by other components. For an ultra-small image sensor that is 1.0±0.3 mm or less, the noise caused by the periodic clock signal as transmitted by the clock line 160 becomes prominent, as disclosed further below.

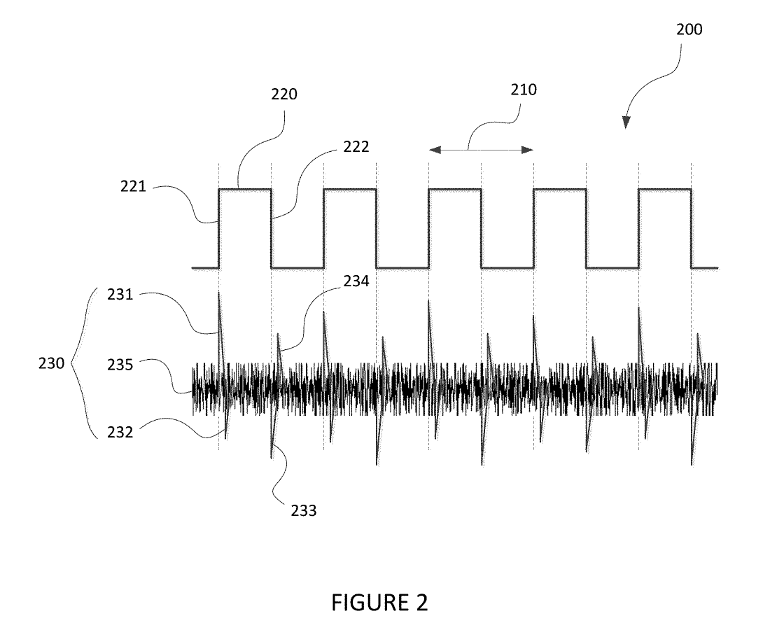

FIG. 2 shows a pulse-train clock signal causing noise to an image data signal. More specifically, signal waveform representation 200 includes a clock signal waveform 220 and a data signal waveform 230. The clock signal waveform 220 may be sent by the supporting module 170 to the image sensor 110 via the clock transmission line 160, as shown in FIG. 1. The data signal waveform 230 may be sent from the image sensor 110 to the supporting module 170 via the data/control signal transmission line 130, as shown in FIG. 1. The clock signal waveform 220 is generally a periodic signal with a fixed period T, which is represented by the double arrowed line segment 210 in FIG. 2. This periodic clock signal 220 may be exemplified by a pulse-train series of square waves. In FIG. 2, the clock signal 220 appears to have a duty cycle of 50%, which means that each pulse occupies 50% of the fixed period T. Other duty cycles, such as 25% or 75% may also be viable. As shown in FIG. 2, each clock pulse includes a rising clock edge 221 and a falling clock edge 222.

The data signal waveform 230 includes a data signal 235 and a series of sharp, spike-like peaks. It is appreciated that these sharp peaks are noise that are caused by the clock pulses 220. More specifically, at each rising clock edge 221, there is a corresponding positive sharp peak 231, a corresponding negative sharp peak 232, or both, that occur at the digital waveform 230. At each falling clock edge 222, there is a corresponding negative sharp peak 233, a corresponding positive sharp peak 234, or both, that occur at the digital waveform 230. These sharp peaks that correspond with the rising and falling clock edges are confirmed to be noise that are associated with the clock signal transmission line 130, because if the clock line 130 is eliminated, then these noise peaks will be significantly reduced or disappear altogether.

As long as there is a electrical clock signal that is needed for timing operations to drive the sequential digital logic that is needed for imaging and image processing, such an electrical clock signal will likely produce its periodic noise to some degree. For an ultra-small image sensor, this clock related noise is especially pronounced. This clock-related periodic noise affects the image data signal to create dark stripes in output images, and is an undesirable feature that needs to be eliminated.

Several factors exacerbate the clock-related periodic noise. First, since the dimension of the ultra-small image sensor is only about 1.0±0.3 mm, the circuit components are placed very close to each other, so the image producing components are significantly affected by the clock components. Second, the conductor core and shield of the clock line contribute to the noise. It is appreciated that medical endoscopes only have transmission lines that are generally less than two meters, but even this relatively short distance is sufficient to exacerbate the clock noise problem for the ultra-small image sensor. Shortening the length of the clock line will reduce the clock noise, but will also negatively affect the utility of the medical endoscope.

Electronic noises are conventionally removed with filters. The primary source of the aforementioned noise is the clock transmission line 160 itself (see FIG. 1). Therefore, a filter used to remove this noise should be placed inside the image sensor 110, which is the receiving end of the clock noise. However, due to the ultra-small dimension (e.g., 1.0±0.3 mm or less) of the image sensor 110, the conventional approach of using a filter to remove noise is very difficult, if impossible to implement. It is desirable to find an alternative solution to the aforementioned clock-related noise problem.

Second Image Sensor System Embodiment

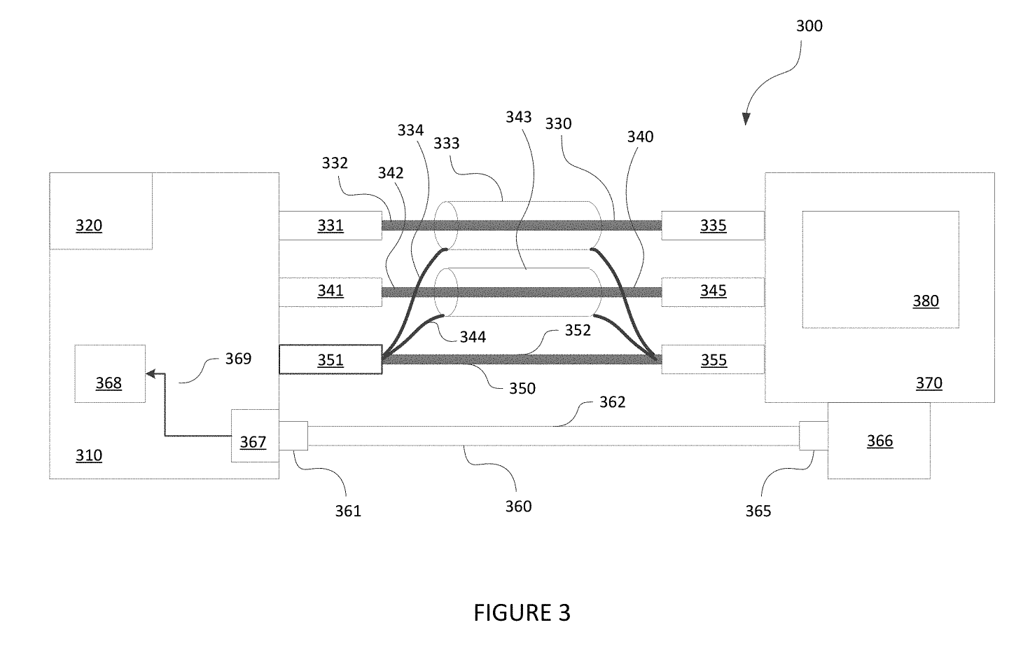

FIG. 3 is a schematic drawing of a second embodiment of an ultra-small image sensor system 300, in which an ultra-small image sensor 310 is coupled with a supporting module 370 by a number of electrical and optical transmission lines. It is similar to the first embodiment of the image sensor system 100 in FIG. 1, but includes improvements.

In FIG. 3, the second embodiment of the ultra-small image sensor 310 has a packaged width of about 1 mm×1 mm, for example, 1.0±0.3 mm in diameter or diagonal dimension. It includes an image sensing member 320 that is, for example, an array of image sensing pixels. Each pixel includes a photodiode to convert photons to charge carriers, a floating node to temporarily store the charge carries, and a number of transistor gates (transfer gate, source follower, reset transistor, etc.) to convey the charge carriers out of the pixel to be further processed by a peripheral circuitry, which is part of the image sensor 310 that surrounds the image sensing pixel array 320.

Due to the ultra-small dimension of the image sensor 310, much of its control and data signal processing function needs to be performed elsewhere outside the sensor, for example, by the supporting module 370, which may perform as a controller, a power source, and other supporting function blocks. More particularly, the supporting module 370 may include a bridge 380, which includes functional blocks such as analog-digital converter (ADC), decoder, image signal processor (ISP), etc.

Similar to the first embodiment of the image sensor system 100 in FIG. 1, for the second embodiment of the image sensor system 300 in FIG. 3, to maintain the ultra-small dimension of the image sensor 310, its circuitry needs to be minimized. For example, only circuits directly relating to image sensing are kept inside the image sensor 310. Circuits that perform other functions are placed outside the image sensor 310, i.e., they are placed inside the supporting module 370. In addition, the transmission lines that feed into the image sensor 310 are minimized to have only four lines. Specifically, there is a power line 340, a ground line 350, a data/control signal line 330, and a clock line 360. The ultra-small dimension of the image sensor 310 entails that only four transmission lines may be accommodated by it. There is no room for additional transmission lines going into the image sensor 310.

FIG. 3 shows specifically that, for the ground line 350, the image sensor 310 includes a sensor-side ground line coupler 351, and the supporting module 370 includes a module-side ground line coupler 355. The ground line 350 connects the two couplers 351 and 355, and includes essentially a conductor line 352 that may be made of electrically conductive material such as copper, or other metals.

FIG. 3 also shows that, for the data/control signal transmission line 330, the image sensor 310 includes a sensor-side data/signal transmission line coupler 331, and the supporting module 370 includes a module-side data/signal transmission line coupler 335. The data/control signal line 330 connects the two couplers 331 and 335. The data/control signal line 330 is shown to be a co-axial cable that includes a line core 332 and a line shield 333. Transmission line types other than the co-axial cable may also be viable, e.g., a twisted pair line. The shield 333 is connected to the ground line 350 with one or a number of shield grounding lines 334. This grounding is desirable, but it is also optional.

Similarly, for the power line 340, the image sensor 310 includes a sensor-side power line coupler 341; the supporting module 370 includes a module-side power line coupler 345. The power line 340 connects the two power line couplers 341 and 345. The power line 340 is shown to be co-axial cables that each includes a line core 342 and a line shield 343. Transmission line types other than the co-axial cable may also be viable, e.g., a twisted pair line. The power line shield 343 is connected to the ground line 350 with one or a number of shield grounding lines 344 respectively. This grounding is desirable, but it is also optional.

For the clock line 360, FIG. 3 shows that it is based on fiber optics instead of the conventional transmission line design that is based on conducting electricity. More specifically, the image sensor 310 includes a sensor-side clock line coupler 361; the supporting module 370 is coupled to a clock signal light source 366, which is in turn coupled to a module-side clock line coupler 365. Conventional electrical clock signals, e.g., in the form of a pulse train of square waves as shown in FIG. 2, are still generated within the supporting module 370, and is used to drive the clock signal light source 366 to emit corresponding light pulses in a digital format, e.g., high/low, or on/off. Such a pulsed light signal is now treated as the clock signal, in place of the more conventional electrical clock signal in a digital voltage form. Alternatively, the pulsed light signal may be provided by the light source 366 all by itself, without interfacing with the supporting module 370.

The pulsed light clock signal is transmitted from the light source 366 through the module-side clock coupler 365 to an optical fiber 362, which is further transmitted through the optical fiber clock line 362 and the sensor-side clock coupler 365 into the image sensor 310. The optic fiber clock line 362 connects the two clock line couplers 361 and 365. It includes a flexible, transparent fiber made of a glass (silica) core, and is wrapped by a transparent cladding material that has a lower index of refraction than that of the glass core. The optic fiber clock line 362 is not made of an electric conductor, and does not function like a conventional electrical transmission line.

Inside the image sensor 310, the pulsed light clock signal is converted into an electrical (voltage) clock signal by an opto-electric conversion unit, such as a photodiode 367. The converted voltage clock signal is then transmitted through a clock signal conveyance path 369 to reach an opto-electric supporting block 368 to be further refined. For example, the supporting block 368 may include a phase locked loop (PLL) circuitry that refines the shape of the voltage clock signal to produce a cleaner and more uniform square waveform. The final voltage clock signal, either with or without the aforementioned refinement, supports a necessary part for the timing operation to drive the sequential logic circuitry of the image sensor system 300.

In another example, the data/control signal line 330 may also be based on fiber optics instead of the conventional conductor based transmission line. In such an example, the clock line 360 may be either based on fiber optics or conventional transmission line. Also in such an example, there are opto-electric conversion units (not shown) that are in either or both of the supporting module 370 and the image sensor 310 to convert the data/control signals first from electrical to optical form, and then from optical to electrical form.

It is appreciated that both the clock line 360 and the data/control line 330 are about two meters or less. This is the conventional length of an endoscope line. For the ultra-small image sensor 310, this relatively short length contributes to the clock-related noise. Implementing these lines with fiber optics solves the noise problem without negatively affecting the utility of the endoscope system 300 (e.g., reduction of utility associated with shortening the transmission lines).

Third Image Sensor System Embodiment

The image sensor system 300 shown in FIG. 3 is further augmented to include illumination components, to be packaged into a final product version for applications such as medical endoscopy. Designed to work inside a dark environment such as digestive tract or blood vessel, an endoscope needs its own illumination means, i.e., its own light source, while not relying on ambient light for illumination.

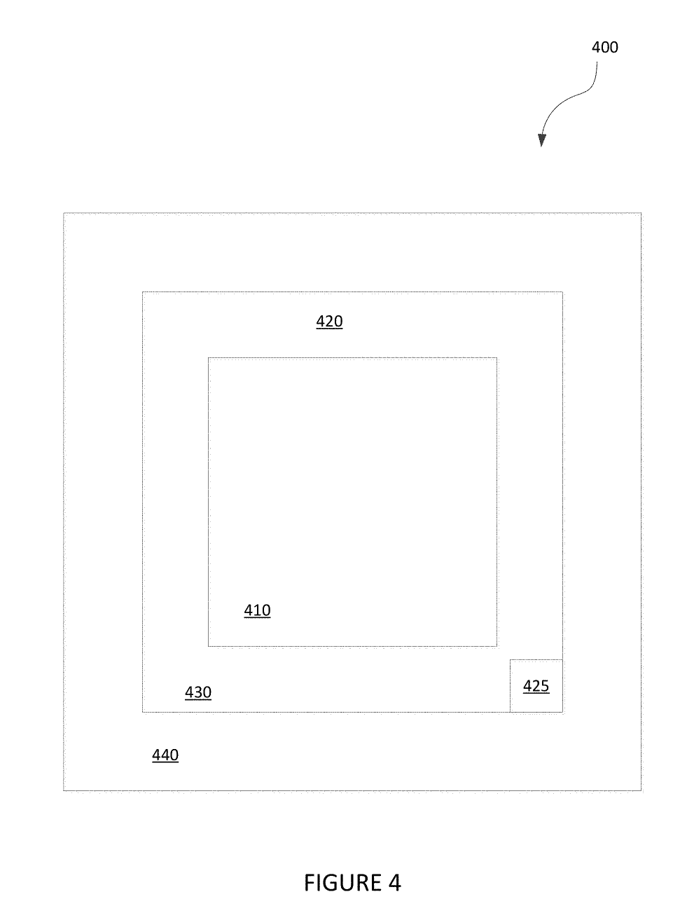

FIG. 4 is a front side view of a variation embodiment of the ultra-small image sensor system 300, which is augmented with illumination components. A third ultra-small image sensor system package 400, e.g., an endoscope, includes a sensor pixel array 410 at its center. The sensor pixel array 410 is similar to the sensor pixel array 320 in FIG. 3. It is surrounded by a peripheral region of supporting circuitry 420, which is necessary for the sensor pixel array 410 to function. The sensor pixel array 410 and the peripheral region of supporting circuitry 420 make up a sensor die 430, as shown in FIG. 4. The sensor die 430 has an ultra-small dimension, e.g., 1.0±0.3 mm. The peripheral region 420 has an exemplary width of around 90 micrometers. A part of the sensor die 430, e.g., in the peripheral region 420, is occupied by an opto-electric conversion unit 425, which is similar to the opto-electric conversion unit 367 in FIG. 3. This may be a photodiode, and may have an exemplary dimension of about 50×50 micrometers. Surrounding the sensor die 430 is a peripheral region 440 packed with fiber optic for illumination. This fiber optic peripheral region 440 may have an exemplary width of about 0.5 mm. The overall footprint of the ultra-small image sensor system package 400 is around 2.00.5 mm.

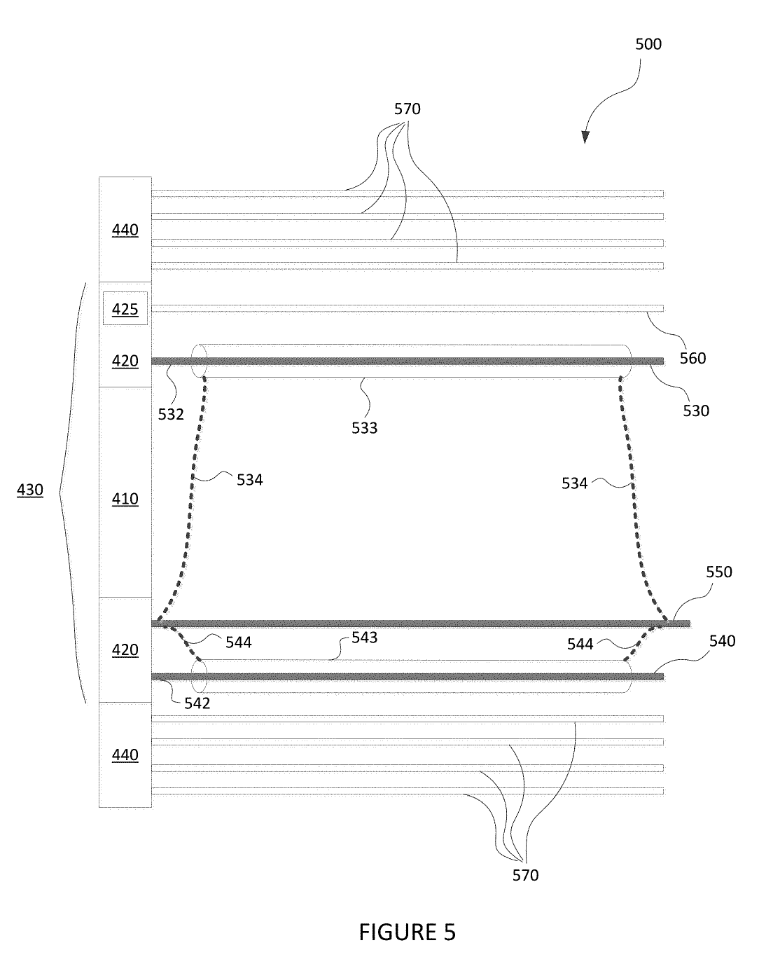

FIG. 5 is a cross sectional side view of a portion of the variation embodiment of the ultra-small image sensor system 300, which is augmented with illumination components. It is the same as the third embodiment of an image system package 400 in FIG. 4, but is now shown as a cross sectional side view. In FIG. 5, a third ultra-small image sensor system package 500 includes the sensor die 430, which includes the pixel array 410, the peripheral region of supporting circuitry 420, the opto-electric conversion unit 425, and the fiber optic peripheral region 440. These components have been previously disclosed in relation to FIG. 4.

Also shown in FIG. 5 is a ground line 550 connected to the peripheral region of supporting circuitry 420. The ground line 550 is similar to the ground line 350 as previously disclosed in FIG. 3. Notably, the previously disclosed couplers 351 and 355 are not shown in FIG. 5, but may still be present. Also shown is a data/control signal line 530 that is connected to the peripheral region of supporting circuitry 420. The data/control signal line 530 is similar to the control/data signal line 330 as previously disclosed in FIG. 3. Notably, the previously disclosed couplers 331 and 335 are not shown in FIG. 5, but may still be present. Here, the data/control line 530 is depicted as a co-axial cable transmission line, with a line core 532 and a line shield 533, wherein the line shield 533 is optionally connected to the ground line 550 with grounding lines 534 (dotted lines).

Also shown is a power line 540 connected to the peripheral region of supporting circuitry 420. The power line 540 is similar to the power line 340 as previously disclosed in FIG. 3. Notably, the previously disclosed couplers 341 and 345 are not shown in FIG. 5, but may still be present. Here, the power line 540 is depicted as a co-axial cable transmission line, with a line core 542 and a line shield 543, wherein the line shield 543 is optionally connected to the ground line 550 with grounding lines 544 (dotted lined).

In FIG. 5, a fiber optic clock line 560 is connected to the peripheral region of supporting circuitry 420. More particularly, it is connected to the opto-electric conversion unit 425. The clock line 560 is similar to the clock line 360 as previously disclosed in FIG. 3. Notably, the previously disclosed couplers 361 and 365 are not shown in FIG. 5, but may still be present. The fiber optic clock line 560 may have a diameter of about 9 to 60 micrometers, and has a length of about two meters or less. The opto-electric conversion unit 425 may have a dimension of about 50×50 micrometers, as previously disclosed.

A number of optical illumination fibers 570 are connected to the fiber optic peripheral region 440 at one end. At the other end, these optical illumination fibers 570 are connected to light sources, e.g., LEDs (not shown). This design functions to provide illumination for the ultra-small image sensor system package 500. The optical illumination fibers 570 may have a diameter of about 9 to 60 micrometers.

In another example, the data/control signal line 530 may also be based on fiber optics instead of the conventional conductor based transmission line. In such an example, the clock line 560 may be either based on fiber optics or conventional transmission line. Also in such an example, there are opto-electric conversion units (not shown) that are in either or both of the supporting module 370 (see FIG. 3) and the image sensor 310 (see FIG. 3) to convert the data/control signals first from electrical to optical form, and then from optical to electrical form.

The abovementioned examples disclose the pixel array 410 in the same plane as the supporting circuitry 420. In an alternative example, the pixel array 410 and the supporting circuitry 420 are stacked in order to reduce the image sensor footprint. More specifically, the supporting circuitry 420 positioned at and couple to the backside of the pixel array 410. The four transmission lines are still connected to the supporting circuitry 420 as previously disclosed.

The above description of illustrated examples of the invention, including what is described in the Abstract, is not intended to be exhaustive or to limit the invention to the precise forms disclosed. While specific examples of the invention are described herein for illustrative purposes, various modifications are possible within the scope of the invention, as those skilled in the relevant art will recognize.

These modifications can be made to the invention in light of the above detailed description. The terms used in the following claims should not be construed to limit the invention to the specific examples disclosed in the specification. Rather, the scope of the invention is to be determined entirely by the following claims, which are to be construed in accordance with established doctrines of claim interpretation.

What is claimed is:

1. An image sensor system, comprising:
(a) an image sensor including an image sensing pixel array;
(b) a supporting module;
(c) a ground line coupling the image sensor with the supporting module;
(d) a power line coupling the image sensor with the supporting module;
(e) a data/control line coupling the image sensor with the supporting module; and
(f) a clock line coupling the image sensor with the supporting module;
wherein the ground line and the power line are based on electrical conduction, and wherein the clock line is based on fiber optics.

2. The image sensor system of claim 1, wherein the data/control line is based on electrical conduction.

3. The image sensor system of claim 1, wherein the data/control line is based on fiber optics.

4. The image sensor system of claim 1, wherein the supporting module further comprises a light source.

5. The image sensor system of claim 4, wherein the clock line is coupled between the light source and the image sensor.

6. The image sensor system of claim 5, wherein the image sensor includes an opto-electric conversion unit, and wherein the clock line is coupled between the opto-electric conversion unit and the light source.

7. The image sensor system of claim 6, wherein the image sensor further includes an opto-electric support unit that is coupled to the opto-electric conversion unit.

8. The image sensor system of claim 7, wherein the opto-electric support unit is a phase locked loop circuit.

9. The image sensor system of claim 4, wherein the supporting module is configured to generate a pulse-train clock signal, wherein the pulse-train clock signal is conveyed to the light source to generate a pulsed light signal.

10. The image sensor system of claim 9, wherein the image sensor has width of no more than about 1.3 millimeters.

11. The image sensor system of claim 10, wherein there are no lines coupling the image sensor and the supporting module, other than the ground line, the power line, the data/control signal line, and the clock line.

12. The image sensor system of claim 10, wherein the image sensor includes a peripheral circuit region that surrounds the image sensing pixel array.

13. The image sensor system of claim 12, wherein the opto-electric conversion unit is situated within the peripheral circuit region.

14. The image sensor system of claim 13, further comprising a fiber optic illumination region that surrounds the peripheral circuit region.

15. The image sensor system of claim 14, further comprising a multitude of illumination optical fibers that are connected to the fiber optic illumination region.

16. An endoscope system, comprising:
(a) an image sensor including an image sensing pixel array;
(b) a peripheral circuit region that surrounds the image sensing pixel array;
(c) a fiber optic illumination region that surrounds the peripheral circuit region;
(d) a supporting module;
(e) a ground line coupling the image sensor with the supporting module;
(f) a power line coupling the image sensor with the supporting module;
(g) a data/control line coupling the image sensor with the supporting module;
(h) a clock line coupling the image sensor with the supporting module; and
(i) a multitude of illumination optical fibers that are coupled to the fiber optic illumination region;
wherein the ground line and the power line are based on electrical conduction, and wherein the clock line is based on fiber optics.

17. The endoscope system of claim 16, wherein the data/control line is based on fiber optics.

18. The endoscope system of claim 16, wherein the supporting module further comprises a light source, wherein the image sensor further includes an opto-electric conversion unit situated within the peripheral circuit region, and wherein the clock line is coupled between the opto-electric conversion unit and the light source.

19. The endoscope system of claim 18, wherein the image sensor has width of no more than about 1.3 millimeters.

20. The endoscope system of claim 19, wherein there are no lines coupling the peripheral circuit region and the supporting module, other than the ground line, the power line, the data/control signal line, and the clock line.
